(19) United States
(12) Patent Application Publication
(10) Pub. No.: US 2023/0081465 A1
(43) Pub. Date: Mar. 16, 2023

(54) TIBETAN FOUR-INGREDIENT LUNG-CLEARING MIXTURE AND APPLICATION THEREOF IN PREPARING MEDICATION FOR TREATING RESPIRATORY DISEASES

(71) Applicant: THE SECOND XIANGYA HOSPITAL OF CENTRAL SOUTH UNIVERSITY, Changsha (CN)

(72) Inventors: SHANGJIE WU, CHANGSHA (CN); SI LEI, CHANGSHA (CN); SHUANGLYU DUAN, CHANGSHA (CN); FEI PENG, CHANGSHA (CN); QUAN ZHANG, CHANGSHA (CN)

(21) Appl. No.: 17/860,079

(22) Filed: Jul. 7, 2022

(30) Foreign Application Priority Data
Sep. 10, 2021 (CN) 202111058544X

(51) Int. Cl. A61K 36/45; A61K 36/515; A61K 36/29; A61K 36/505; A61P 11/00

(52) U.S. Cl. CPC A61K 36/45; A61K 36/515; A61K 36/29; A61K 36/505; A61P 11/00

(57) ABSTRACT

The application belongs to the field of natural medicines, and particularly relates to a Tibetan four-ingredient lung-clearing mixture, its raw material consists of Rhododendron anthopogonoidesoides, Gentiana flower, Berberis bark and Herba Corydalis to obtain extracts thereof as an active ingredient. The combination of Rhododendron anthopogonoidesoides, Gentiana flower, Berberis bark and Herba Corydalis could realize synergy effects in effectively improving the anti-inflammatory effects and treatment effects of chronic obstructive pneumonia, and could also improve the lung injury repair function.

### CROSS-REFERENCE TO RELATED APPLICATION

The present application claims priority to Chinese Patent Application No. 202111058544.X, filed on Sep. 10, 2021, the contents of which are hereby incorporated by reference.

### TECHNICAL FIELD

The present application belongs to the technical field of medicine, and particularly relates to a Tibetan medication prescription for respiratory system diseases.

### BACKGROUND

The prevalence of respiratory diseases such as chronic obstructive pulmonary disease (COPD) and lung cancer increases with age, whilst lung infections, as a direct cause of death, still top the list of infectious diseases among the elderly who are prone to aspiration pneumonia due to the low immune function of the body in old age, although various new antibiotics have been introduced.

Moreover, chronic respiratory diseases, which often include COPD, bronchial asthma, bronchiectasis and interstitial lung disease, are the main threats to human life and quality of their living; the diseases are mostly caused by long-term respiratory inhalation of smoke or other harmful particles, and the diseases are particularly likely to occur in individuals with a weakened innate respiratory immune system. Among them, COPD is one of the diseases with the highest morbidity and mortality rates worldwide. COPD is a common respiratory disease characterized by incomplete and progressive airflow limitation, and is one of the leading causes of death worldwide. With incidence rates increasing each year, COPD is expected to become the third leading cause of death worldwide by 2030. It is estimated by the World Health Organization that 80 million people worldwide have COPD of moderate to severe severity, where patients suffer from severe disabilities to work and poor quality of life, resulting in a huge financial burden on society and patients' families. In China, the prevalence of COPD among adults is as high as 8.2%. COPD is characterized by primary symptoms of lung diseases in the early stages and right heart and lung failure in the later stages, in addition to dangerous prognosis and increasing incidence with age.

Chinese Tibetan medicine has flourished for thousands of years with emphases e on the “holistic concept” and “dialectical treatment” and the importance of coordination and balance between the various organs of the patient, and it has been proved to be very effective in treating many diseases with few adverse effects. Developed in plateau region with high altitudes and low oxygen, Tibetan medicines are unique in their therapeutic advantages due to the harsh living environment. As one of the Tibetan medications, the Ten-Ingredient Gentiana Flower Prescription comprises 10 Chinese medicinal materials such as Rhododendron anthopogonoidesoides, Gentiana flower, Herba Corydalis, Berberis bark, and Przewalskia tangutica; the prescription integrates the Tibetan medicine's experience in treating respiratory system diseases and is effective in treating acute bronchitis, infantile pneumonia, community-acquired pneumonia, acute exacerbation of COPD and bronchial asthma, etc., with the effects of clearing heat and reducing inflammation, relieving cough and asthma. It is indeed a treasure of Chinese traditional medicine culture. [1]

Existing medicaments for treating COPD comprise mainly bronchodilators, hormones and that like; however, such conventional agents offer unsustainable nebulised effects and have toxic side effects that are difficult to tolerate.

[1] N. GENG et al., PM2.5 in an industrial district of Zhengzhou, China: Chemical composition and source apportionment, Particuology 11, 2013, pp. 99-109.

### SUMMARY

In order to solve the problems related to the treatment of existing diseases, a brand new Tibetan four-ingredient lung-clearing (also referred to lung-treating) mixture is provided in one of the aspects of the present application with aims of simplifying the components of medicinal materials and reducing side effects of the medication, so as to further improve the drug effect and develop brand new applications.

An application/use of the Tibetan four-ingredient lung-clearing mixture in preparing a medicament for treating respiratory diseases is provided in a further aspect of the present application.

A medicament for treating respiratory diseases by taking the Tibetan four-ingredient lung-clearing mixture as an active ingredient is provided in another aspect of the present application.

The Tibetan four-ingredient lung-clearing mixture, its raw material is composed of Rhododendron anthopogonoides, Gentiana flower, Berberis bark and Herba Corydalis and thereby extracts thereof are obtained to get the lung-clearing mixture may be for oral administration.

It is found in present application that the combination of Rhododendron anthopogonoidesoides, Gentiana flower, Berberis bark and Herba Corydalis can be synergized to effectively improve the anti-inflammatory aspects and the therapeutic effects in treating COPD as well as improving the reparative function of the lung. It is also found in present application that compared with that ten medicinal materials composed of Gentiana flower, Rhododendron anthopogonoidesoides, liquorice, Herba Corydalis, Fritillaria cirrhosa, Berberis bark, Codonopsis convolvulacea Kurz, Phlomis younghusbandii Mukerjee, Radix Inulae Racemosae and Przewalskia tangutica, the Tibetan four-ingredient lung-clearing mixture disclosed by the present application has significant omission of ingredients, such that not only does it not lead to functional annihilation, but on the contrary, it unexpectedly achieves significant synergistic effects in improving the anti-inflammatory effect in addition to good injury-repairing capability of lung, which can be used for the treatment of acute lung injury; moreover, the drug described in the present invention is able to reduce the side effects of administration, apart from its significant pharmacological enhancement.

In the present application, the medicinal part of Rhododendron anthopogonoidesoides is flowers and/or leaves, preferably the flowers and/or leaves of Rhododendron cephalanthum Franch. of Ericaceae family;

the medicinal part of Gentiana flower is flowers, preferably flowers of Gentiana szechenyii Kantiz. of Gentianaceae family;

the medicinal part of Herba Corydalis is whole plant, preferably whole plant of Corydalis mucronifera Maxim. of Papaveraceae family;

the medicinal part of Berberis bark is bast, preferably inner bast of stem or root of Berberis dasystachya Maxim. of Berberidaceae family.

In the present application, the used medicinal materials can be fresh medicinal materials, dried medicinal materials, or medicinal material drinks.

Preferably, the medicinal materials include in parts by weight of: 100-600 parts of Rhododendron anthopogonoidesoides, preferably 400-500 parts; 60-120 parts of Gentiana flower, preferably 60-100 parts; 60-120 parts of Herba Corydalis, preferably 60-100 parts; 100-550 parts of Berberis bark, preferably 400-500 parts.

Preferably, that extracts include a volatile oil of Rhododendron anthopogonoidesoides and water extracts of the raw material. The extracts can be prepared by the method as follow:

steam distilling Rhododendron anthopogonoidesoides to obtain volatile oil, a water extract A and a medicinal residue; decocting the residue, the Gentiana flower, the Berberis bark and the Herba Corydalis in water to obtain a water extract B, and mixing the volatile oil and the water extracts A and B to obtain the resultant extracts.

The present application also provides an application of the Tibetan four-ingredient lung-clearing mixture for preparing a medicament for treating respiratory diseases.

It is found that the disclosed Tibetan four-ingredient lung-clearing mixture can realize synergy effect in improving the anti-inflammatory effect and treatment effect of COPD with excellent performance in treating respiratory system disease; it can also be utilized in treating acute lung injury with effects beneficiary to injury-repairing capability of the lung.

As for the application of the Tibetan four-ingredient lung-clearing mixture disclosed in present application, the respiratory system disease refers to at least one of COPD and acute lung injury.

Under a preferred application, the extracts of the Tibetan four-ingredient lung-clearing mixture are obtained and prepared into a pharmaceutical preparation used for nebulised administration via the respiratory system.

It is found in present application that the extracts of the prepared nebulised pharmaceutical preparation of the obtained from the Tibetan four-ingredient lung-clearing mixture can further improve the therapeutic performance in treating COPD and acute lung injury, and help to reduce the side effect of the medicine.

The present application also provides a medicament for treating respiratory system diseases, which takes the Tibetan four-ingredient lung-clearing mixture with a pharmaceutically effective amount as an active ingredient.

The medicament disclosed above includes the following active components/ingredients: extracts of a raw material consisting of the Rhododendron anthopogonoidesoides, the Gentiana flower, the Berberis bark and the Herba Corydalis; based on the active ingredients described, the medicament enables the treatment of respiratory diseases.

The medicament for treating respiratory system diseases is an atomizing agent (also referred to aerosol liquid) for nebulised administration through the respiratory system and comprises the extracts of the Tibetan four-ingredient lung-clearing mixture as active components and nebulizing adjuvant (also referred to auxiliary material for aerosol).

In present application, the active components in the atomizing agent include volatile oil of the Rhododendron anthopogonoidesoides in the Tibetan four-ingredient lung-clearing mixture and water extracts. It is found in the present application that while treating chronic respiratory diseases, this medicine can significantly slow down the process of acute lung injury by reducing the inflammatory response and inhibiting the release of a large number of inflammatory cells and cytokines, thus improving lung injury and promote lung recovery. By means of topical administration in the respiratory tract and lungs by nebulisation, the active ingredients synergize with each other to cleanse and moisten the airways as well as to reduce the infiltration of inflammatory cells, inhibit the release of cytokines such as interleukins and ameliorate the narrowing of the airways, thus significantly enhancing the therapeutic effect; moreover, the active ingredients can synergistically reduce the cellular intolerance of topical administration and contribute to a significant reduction in toxicity to the organism.

The nebulizing adjuvant of the present application may be any adjuvant known to facilitate nebulization of the active ingredient.

Preferably, that medicament for treating respiratory diseases is an aerosol for treating COPD and/or acute lung injury.

Prescription: four Tibetan medicines of Rhododendron anthopogonoidesoides, Gentiana flower, Herba Corydalis and Berberis bark, where Rhododendron anthopogonoidesoides and Gentiana flower are sovereign drugs to clear heat and eliminate dampness, Herba Corydalis and Berberis bark are ministerial drugs for clearing heat and eliminating phlegm. Indications: clearing away heat and phlegm, and relieving cough and asthma; it can be used for treating cough, wheezing, and yellowish sputum caused by phlegm and heat obstructing lung, or concurrent fever, nasal discharge, sore throat, thirst, yellowish urine, and dry stool; the above symptoms can be seen in acute attacks of acute bronchitis and chronic bronchitis.

### Beneficial Effects

The present application provides a Tibetan four-ingredient lung-clearing mixture, its raw material consists of Rhododendron anthopogonoidesoides, Gentiana flower, Berberis bark and Herba Corydalis and thereby extracts thereof are obtained, and the Tibetan four-ingredient lung-clearing mixture is found to be capable of improving anti-inflammatory activity based on the synergy of the four medicines, and in addition, good lung injury repairing capability can be achieved synergistically.

### BRIEF DESCRIPTION OF DRAWINGS

FIG. 1 shows an experimental diagram of a protective study of COPD in application embodiment 1.

FIGS. 2A-2F illustrate cell levels of BALF (Bronchoalveolar Lavage Fluid) of application embodiment 1.

FIGS. 3A-3B illustrate levels of IL-6, IL-8, and TNF-a in serum and BALF of application embodiment 1.

FIGS. 4A-4B are diagrams showing the lung function of application embodiment 1.

FIG. 5 shows a HE (Hematoxylin and Eosin) staining diagram of lung tissue in application embodiment 1.

FIGS. 6A-6B illustrate results of MLI and D1 of application embodiment 1.

FIGS. 7A-7C are diagrams showing results of cell proliferation detected by CCK-8 in primary mouse type II alveolar epithelial cells in application embodiment 2.

FIGS. 8A-8D are diagrams illustrating expression levels of cytokines IL-6, TNF-a, AngII, and Ang1-7 detected by ELISA (Enzyme-Linked Immuno Sorbent Assay) in primary mouse type II alveolar epithelial cells in application embodiment 2.

FIGS. 9A-9D show expression levels of cytokines IL-6, TNF-a, AGT and Ang1-7 detected by ELISA in LPS-induced acute lung injury in mouse.

FIG. 10 shows a HE staining diagram of the Tibetan medicine in LPS-induced acute lung injury in mouse (HE×400).

### DETAILED DESCRIPTION OF EMBODIMENTS

Rhododendron anthopogonoidesoides may be: dried flower and leaf of Rhododendron cephalanthum Franch. of Ericaceae family; Gentiana flower may be: dried flower of Gentiana szechenyii Kantiz. Of Gentianaceae family; Herba Corydalis may be: dried whole plant of Corydalis mucronifera Maxim. of Papaveraceae family; Berberis bark may be: inner bark of the stem or root of Berberis dasystachya Maxim. of Berberidaceae family.

### Preparation Embodiment 1

A preparation method of the Tibetan four-ingredient lung-clearing mixture may include the following steps:

prescription of the raw materials: Rhododendron anthopogonoidesoides 500 g (gram), Gentiana algida Pall. (Gentiana flower) 100 g, Herba Corydalis 100 g, and Berberis bark 500 g;

weighing Rhododendron anthopogonoidesoides and soaking in distilled water with 7 times the weight of the medicinal material for 1 h (hour), extracting and collecting the volatile oil by steam distillation, sealing and storing the oil in a refrigerator; performing filtration to the Rhododendron anthopogonoidesoides and the distilled water after extracting the volatile oil, and concentrating the filtrate to appropriate amount for later use; adding Gentiana algida Pall. Herba Corydalise and Berberis bark into the residue of Rhododendron anthopogonoidesoides, decocting the four ingredients in water for 2 times, where 10 times of water with respect to the four ingredients by weight is added for the first decocting with decocting duration of 1.5 h, followed by filtering with 200-mesh filter screen; and 8 times of water is added for that second decocting with decocting duration of 1.0 h, followed by filtering with a 200-mesh filter screen; combining the filtrates, concentrating under reduced pressure (at the temperature of 60-70° C., and the vacuum degree is (−0.06)Mpa-(−0.08)Mpa) to obtain a concentrated solution with a relative density of 1.05-1.08 (at 60° C.), and centrifuging to obtain the Tibetan four-ingredient lung-clearing mixture (in the following embodiments, the Tibetan four-ingredient lung-clearing mixture is essentially the extracts prepared by this embodiment unless otherwise stated);

### Comparative Embodiment 1

The process for the reference Rhododendron anthopogonoidesoides in comparative embodiment is as follows:

extraction of Rhododendron anthopogonoidesoides volatile oil: using steam distillation to extract the volatile oil (using distilled water with the amount of 7 times that of the medicinal materials for reflux extraction for 6 hours, and using a small amount of ether to collect the volatile oil, then volatilizing the ether to obtain the final product); the extracted volatile oil of Rhododendron anthopogonoidesoides is a pale yellow oil with a special strong odor, its relative density is less than 1, and the content is 0.74% (mL/g), which is in consistent with the requirements of standard WS3-BC-0078-95 “content determination” for Rhododendron anthopogonoidesoides.

Preparation of water extract of Rhododendron anthopogonoidesoides: adding water into the volatile oil removed Rhododendron anthopogonoidesoides for extraction for 2 times (adding 10 times of distilled water for each time, and performing reflux extraction for 2 h), filtering the extracted solution, filtering, concentrating under reduced pressure at 50° C. to 1 g per mL (milliliter) of Rhododendron anthopogonoidesoides medicinal material, adding volatile oil (mixing the volatile oil and glycerol in a ratio of 1:1), and mixing to obtain water extract of Rhododendron anthopogonoidesoides (concentration of 1 g/mL).

In the process of subsequent experiments, normal saline is used for dilution and nebulization.

### Comparative Embodiment 2

The extraction process of Gentiana flower is as follows:

preparation of water extract of Gentiana flower: extracting Gentiana flower with distilled water for 2 times (adding 14 times of distilled water for each time, and performing reflux extraction for 2 h), mixing the extracted solutions, filtering, and concentrating the filtrate under reduced pressure to obtain Gentiana flower extract with concentration of 0.77 g/mL; the extract is viscous with brownish yellow color and a relatively weak odor; in the process of subsequent experiments, normal saline is used for dilution and nebulization.

### Comparative Embodiment 3

The preparation of the ten-ingredients Gentiana flower compound medicine atomizing agent is as follows:

weight ratio of ten-ingredients Gentiana flower medicinal materials (calculated by dry weight):

500 g Rhododendron anthopogonoidesoides, 100 g Gentiana flower (Gentiana algida Pall.), 500 g Berberis bark, 100 g Herba Corydalis, 10 g Przewalskia tangutica, 50 g licorice, 50 g Fritillaria cirrhosa, 50 g Phlomis younghusbandii Mukerjee, 50 g Codonopsis convolvulacea Kurz and 80 g Radix Inulae Racemosae;

water extraction (group a): weighing Rhododendron anthopogonoidesoides, soaking in distilled water with 7 times the weight of the medicinal materials for 1 h; extracting the volatile oil by steam distillation, collecting the volatile oil, sealing and storing in a refrigerator; filtering the extract, and concentrating the filtrate to appropriate amount for later use; adding Gentiana flower, Berberis bark, Herba Corydalis, Przewalskia tangutica, licorice, Fritillaria cirrhosa, Phlomis younghusbandii Mukerjee, Codonopsis convolvulacea Kurz and Radix Inulae Racemosae into that residue, soaking in distilled water 7 times of the medicinal materials for 1 h, performing reflux extracting for 2 times for 2 h each time, combining the extractive solutions, filtering, concentrating the filtrate under reduced pressure to appropriate amount, adding the concentrated solution of the first extraction of Rhododendron anthopogonoidesoides, continuing to concentrate under reduced pressure to each ml of extract is equivalent to 1 g medicinal materials, adding the volatile oil of Rhododendron anthopogonoidesoides extracted in the previous period, and uniformly mixing to obtain an atomizing agent (1 g/mL); the component comprises at least one of choline, gentiopicrin, peimine, berberine, and glycyrrhetinic acid;

water extraction and alcohol precipitation extraction (group b): taking medicinal materials with that prescription amount of ten-ingredients Gentiana flower, taking Rhododendron anthopogonoidesoides and soaking it with distilled water of 7 times of the medicinal materials for soaking for 1 h; extracting volatile oil by a steam distillation method, collecting the volatile oil, sealing and storing in a refrigerator at 4° C.; filtering to obtain the extracting solution for later use; adding nine medicinal materials of Gentiana flower, Berberis bark, Herba Corydalis, Przewalskia tangutica, licorice, Fritillaria cirrhosa, Phlomis younghusbandii Mukerjee, Codonopsis convolvulacea Kurz and Radix Inulae Racemosae continuously into the Rhododendron anthopogonoidesoides residue, separately adding 12 times, 10 times and 10 times of distilled water and extracting for 2 h, 1.5 h and 1 h respectively, filtering, combining the three extracted solutions and the first extracted solution of Rhododendron anthopogonoidesoides, concentrating under reduced pressure at 60° C. until the relative density is 0.5 g/mL, slowly adding ethanol, stirring until the alcohol content reaches 80%, standing overnight, filtering, concentrating the filtrate under reduced pressure at 60° C. until no alcohol smell is observed, adding the Rhododendron anthopogonoidesoides volatile oil extracted in the previous stage, and uniformly mixing to obtain the ten-ingredients gentian extract (1g/mL).

alcohol extraction (group c): taking the medicinal materials with the prescription amount of ten-ingredients Gentiana flower prescription, adding respectively 12 and 10 times of ethanol to perform reflux extraction for 2 times, filtering, then combining the 2 extracts, concentrating under reduced pressure at 60° C. until the relative density is 1.10 to obtain the alcohol extract of the ten-ingredients Gentiana flower prescription (1 g/mL).

Part I: Study on the Protective Effect of “Tibetan Four-Ingredient Lung-Clearing Mixture” on COPD (Application Embodiment 1)

1. Methodology

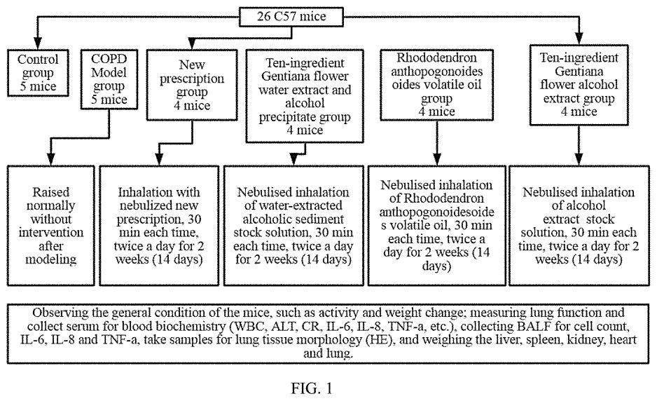

Experimental method is shown in FIG. 1;

a total of 26 C57 mice are grouped into six groups: (1) control group, (2) COPD model group, (3) new prescription group (“Tibetan four-ingredient lung-clearing mixture”, the group of embodiment 1, also known as the new prescription group), (4) Rhododendron anthopogonoidesoides volatile oil group (Group of Comparative embodiment 1), (5) ten-ingredient Gentiana flower water extract and alcohol precipitate group (also referred to as the water extract and alcohol precipitate group in the following parts, Group b of Comparative embodiment 3), and (6) ten-ingredient Gentiana flower alcohol extract group (also referred to as the alcohol extract group in the following parts, Group C of Comparative embodiment 3). The mice in the control group are raised normally without intervention, and mice is other four groups are subjected to smoking treatment, where the smoking condition is as follows: custom-made smoke box of 1.2*0.8*0.8 m in which 5 cigarettes (Acacia cigarettes) are smoked for 30 min at a time, 4 times a day, 5 days a week, for 16 weeks in total. After modeling, the mice in the COPD model group are raised normally without other intervention. The new prescription group, the Rhododendron anthopogonoidesoides volatile oil group, and the water extract and alcohol extract of ten-ingredient Gentiana flower water extract and alcohol precipitate group and ten-ingredient Gentiana flower alcohol extract group are separately administered with the atomizing agent inhalation of the herbal extract of the new prescription, the volatile oil extract of Rhododendron anthopogonoidesoides (1.33% g/mL), the water extract and alcohol extract of ten-ingredient Gentiana flower (1 g/mL), and the alcohol extract of ten-ingredient Gentiana flower (1 g/mL). The specific treatment is conducted for 30 min each time, twice a day, two weeks (14 days) in total. Note: the atomized solvent is normal saline.

2. Results

2.1 General Situation

C57 mice show no obvious toxic reaction during smoking and drug atomization, no obvious abnormality during feeding, and no mouse death or other toxic reaction.

2.2 Comparison of Relevant Indicators

2.2.1 Body Weight Change

The body weights of the mice in the five groups increase, but the difference is not statistically significant (p>0.05) compared to the control group.

TABLE 1
Body weight changes of mice after
multiple atomizing agent inhalation

| | Initial | Final | Weight |
|---|---|---|---|
| | weight | weight | gaining |
| Control group | 20.4 ± 0.37 | 28.45 ± 0.99 | 8.05 ± 1.28 |
| COPD model group | 20.38 ± 0.32 | 28.55 ± 1.12 | 8.17 ± 1.16 |
| New prescription group | 20.23 ± 0.38 | 28.5 ± 0.67 | 8.27 ± 0.72 |
| Water extraction and alcohol | 19.92 ± 0.6 | 28.8 ± 0.7 | 8.88 ± 0.97 |
| precipitation group | | | |
| Alcohol extraction group | 19.94 ± 0.92 | 28.56 ± 0.91 | 8.62 ± 1.68 |
| Volatile oil of | 20.49 ± 0.95 | 28.68 ± 0.72 | 8.19 ± 1.19 |
| Rhododendron | | | |
| anthopogonoidesoides group | | | |

2.2.2 Blood Biochemical Indicators

WBC in COPD model group is higher than that in the control group (p<0.01). The levels of components in the new prescription group and the water extract and alcohol precipitate group are lower than that in the COPD model group (p<0.05). There is no statistical difference between the COPD model group and the alcohol extract group and the volatile oil of Rhododendron anthopogonoidesoides group (p>0.05).

ALT and CR of mice in the COPD model group, the new prescription group, the water extract and alcohol precipitate group, the alcohol extract group, and the Rhododendron anthopogonoidesoides volatile oil group are not significantly different from those in the control group (p>0.05).

TABLE 2
Blood biochemical indicators of mice in
atomizing agent inhalation experiment

| | WBC | ALT | CR |
|---|---|---|---|
| | (10⁹/L) | (U/L) | (mg %) |
| Control group | 6.36 ± 0.98 | 35.4 ± 1.7 | 0.69 ± 0.14 |
| COPD model group | 9.13 ± 1.21** | 35.8 ± 3.9 | 0.67 ± 0.13 |
| New prescription group | 6.75 ± 0.97# | 38.3 ± 5.8 | 0.68 ± 0.15 |
| Water extraction and | 6.77 ± 1.02# | 36.8 ± 5.0 | 0.65 ± 0.17 |
| alcohol precipitation | | | |
| group | | | |
| Alcohol extraction group | 8.52 ± 0.97 | 38.8 ± 3.8 | 0.63 ± 0.08 |
| Volatile oil | 7.87 ± 1.46 | 35.25 ± 4.57 | 0.73 ± 0.2.0 |
| of Rhododendron | | | |
| anthopogonoidesoides | | | |
| group | | | |

***Compared with the control group, p < 0.005,
****Compared with the control group, P < 0.001;
#compared with COPD model group, p < 0.05,
##Compared with COPD model group, p < 0.01;

2.2.3 Weight of Each Organ

There is no significant difference in organ weights between the two groups (p>0.05).

TABLE 3
Weight of each organ in mice after multiple times of atomizing agent inhalation

| | Liver/g | Spleen/g | Kidney/g | Heart/g | Lung/g |
|---|---|---|---|---|---|
| Control group | 6.17 ± 1.06 | 0.44 ± 0.16 | 0.94 ± 0.17 | 0.51 ± 0.12 | 0.94 ± 0.16 |
| COPD model group | 6.22 ± 1.16 | 0.4 ± 0.09 | 1.02 ± 0.25 | 0.48 ± 0.11 | 0.97 ± 0.11 |
| New prescription group | 6.13 ± 0.96 | 0.41 ± 0.12 | 0.99 ± 0.16 | 0.5 ± 0.14 | 0.97 ± 0.14 |
| Water extraction and alcohol | 6.65 ± 1.33 | 0.4 ± 0.02 | 0.79 ± 0.23 | 0.45 ± 0.09 | 0.96 ± 0.17 |
| precipitation group | | | | | |
| Alcohol extraction group | 6 ± 1.62 | 0.4 ± 0.12 | 0.9 ± 0.11 | 0.36 ± 0.09 | 0.92 ± 0.15 |
| Volatile oil of Rhododendron | 6.54 ± 0.61 | 0.59 ± 0.41 | 0.9 ± 0.08 | 0.42 ± 0.13 | 1.01 ± 0.26 |
| anthopogonoidesoides group | | | | | |

2.2.4 Levels of IL-6, IL-8 and TNF-a in Serum and BALF

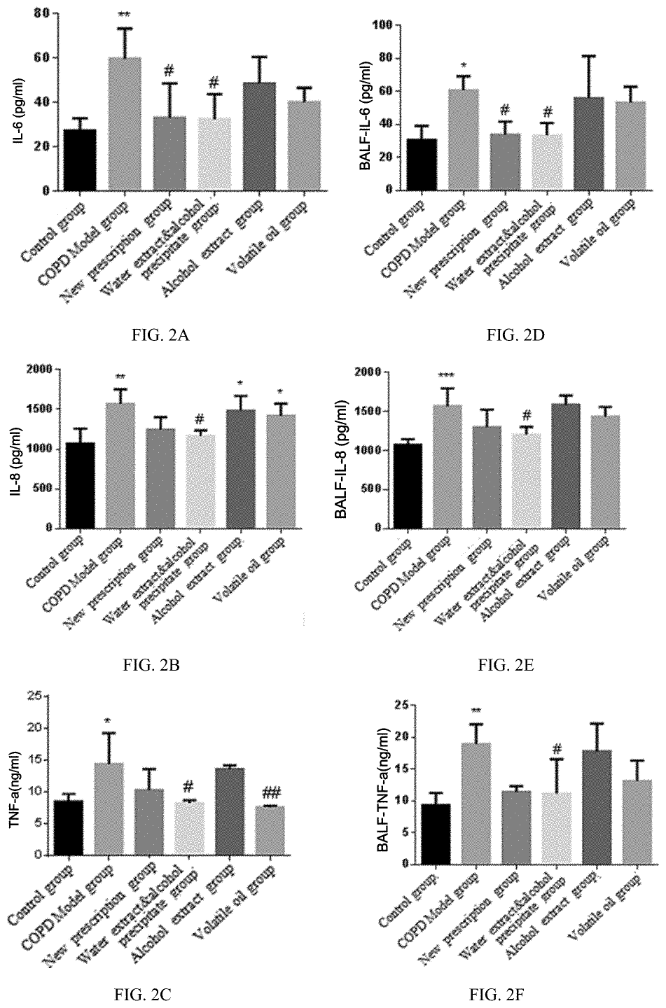

The results are shown in FIGS. 2A-2F.

The levels of IL-6, IL-8 and TNF-a in serum and BALF of patients in the COPD model group are higher than those in the control group (p<0.05). IL-6 in serum and BALF of the new prescription group is lower than that of the COPD model group. IL-6, IL-8, and TNF-a in serum and BALF of the water extract and alcohol precipitate group are all lower than that of the COPD model group (p<0.05). There is no statistical difference in the above indicators between the alcohol extract group and the COPD model group (p>0.05). Serum TNF-a is decreased slightly in the Rhododendron anthopogonoidesoides volatile oil group, and it is also decreased slightly in the COPD model group (p<0.05). However, there is no statistical difference in other indicators between the Rhododendron anthopogonoidesoides volatile oil group and the COPD model group (p>0.05).

2.2.5 Cell Counts in BALF

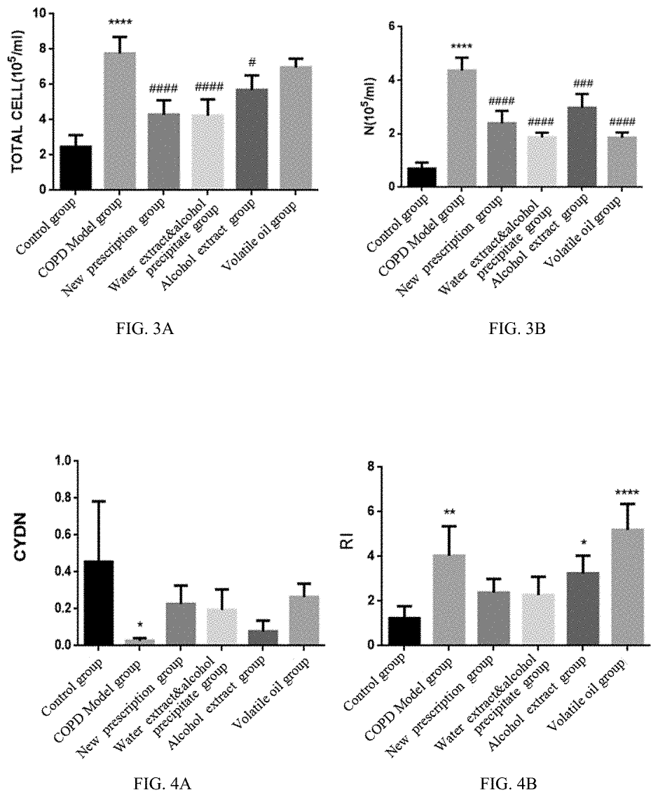

The results are shown in FIGS. 3A-3B. The total cells and neutrophils in BALF of rats in the COPD model group are higher than those in the control group (p<0.05). The total cells in BALF of the new prescription group, the water extract and alcohol precipitate group, and the alcohol extract group are lower than that of the COPD model group. The neutrophils in BALF of the new prescription group, the water extract and alcohol precipitate group, the alcohol extract group, and the Rhododendron anthopogonoidesoides volatile oil group are lower than that of the COPD model group (p<0.05).

2.2.6 Determination of Lung Function

The results are shown in FIGS. 4A-4B. RI of lung functions in the COPD model group is better than that of the control group, while cydn is lower than that of the control group (p<0.05). The increased airway resistance and decreased dynamic pulmonary compliance in the COPD model group indicate the existence of airway airflow limitation in the mice, and the modeling is successful.

2.2.7 HE Results of Lung Tissue

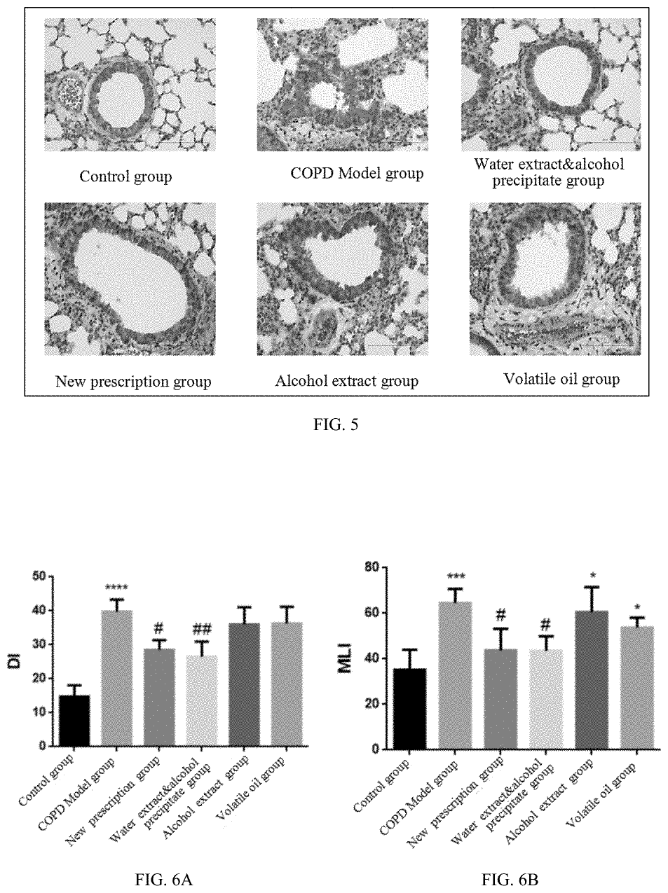

The results are shown in FIG. 5:

In the control group, the airway epithelial is intact with neat cilia, normal alveolar structure and less inflammatory cell infiltration around the airways and blood vessels; in the COPD model group: narrowed and deformed fine bronchial tubes, disorganized cilia, shedding and necrosis of bronchial cup cells, thickening of some tube walls, dilatation of alveolar lumen, septum break and fusion, infiltration of inflammatory cells such as neutrophils and macrophages can be seen around airways and blood vessels. Compared with the COPD model group, the new prescription group shows a significant reduction in alveolar cavity enlargement, cilia disorder and inflammatory cells. In contrast, the above effects are all reduced in the water extract and alcohol precipitate group, the alcohol extract group, and the volatile oil of Rhododendron anthopogonoidesoides group.

MLI and DI in the COPD model group are higher than those in the control group (p<0.05), indicating the successful modeling. The MLI and DI values in the new prescription group and the water extract-alcohol precipitate group are lower than those in the COPD model group (p<0.05). Results are presented in FIGS. 6A-6B.

4. Summary

The extract of the new prescription (Tibetan four-ingredients lung-clearing mixture) also has a good protective effect on mice of COPD.

Part II: Study on the Protective Effect of “Tibetan Four-Ingredients Lung-Clearing Mixture” on Acute Lung Injury (Application Embodiment 2)

In this part, the protective effect of “Tibetan four-ingredients lung-clearing mixture” on respiratory system in acute lung injury is explored; which is a new protective effect. No related effects have been mentioned in the indications of this compound preparation. In the present study, it is found that this product could significantly slow down the process of acute lung injury caused by Lipopolysaccharide (LPS), reduce the inflammatory response, inhibit the release of a large number of inflammatory cells and cytokines, improve lung injury and promote lung recovery.

(1) Protective Effects of Tibetan Medicine on Primary Mouse Type II Alveolar Epithelial Cells

1) Experimental Grouping:

The mice are grouped into a blank control group, an LPS intervention group (LPS), an LPS+normal rat serum group (LPS+S), and LPS+compound Tibetan medicine extract group (the extract obtained in group b of Comparative embodiment 3, also known as Tibetan medicine group 1, labeled as LPS+1), and an LPS+Tibetan four-ingredients extract group (prepared in Embodiment 1, also known as Tibetan medicine group 2, labeled as LPS+2).

Intervention methods are as follows:

The cells in the blank Control group (Control group) are not subjected to any intervention.

LPS intervention group (LPS), treated with LPS (10 ug/mL);

LPS+normal rat serum group (LPS+S), treated with LPS (10 ug/mL)+normal rat serum;

LPS+compound Tibetan medicine extract group (LPS+1) is treated with LPS (10 ug/mL) +compound Tibetan medicine extract (extract of group b of Comparative embodiment 3) rat serum.

LPS+Tibetan four-ingredients extract group (LPS+2) is treated with LPS (10 ug/mL)+Tibetan four-ingredients extract (prepared in Embodiment 1) rat serum.

2) CCK-8 is Used to Detect Cell Proliferation:

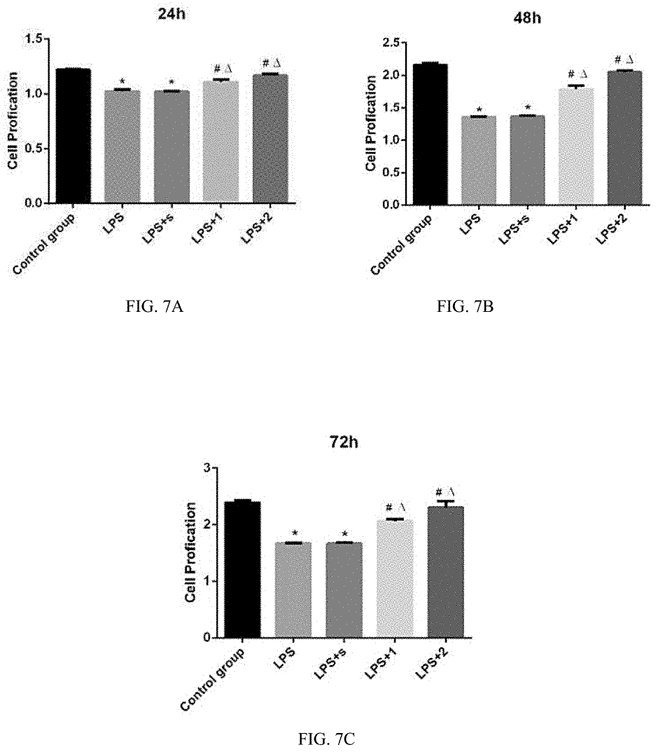

the results show that the cell activities of the LPS and LPS normal serum groups are lower than those of the control group at 24 h, 48 h and 72 h (p<0.05). The cell activities of the LPS+1 group and LPS+2 group are higher than those of the LPS and LPS+s group at 24 h, 48 h and 72 h (p<0.05). Results are presented in FIGS. 7A-7C.

Note: *: compared with the Control group, P<0.05; #: compared with the LPS group, P<0.05; Δ: compared with the LPS+s group, P<0.05. The same below.

3) Detection of the Expression Levels of Cytokines IL-6, TNF-a, AngII, and Ang1-7 by ELISA

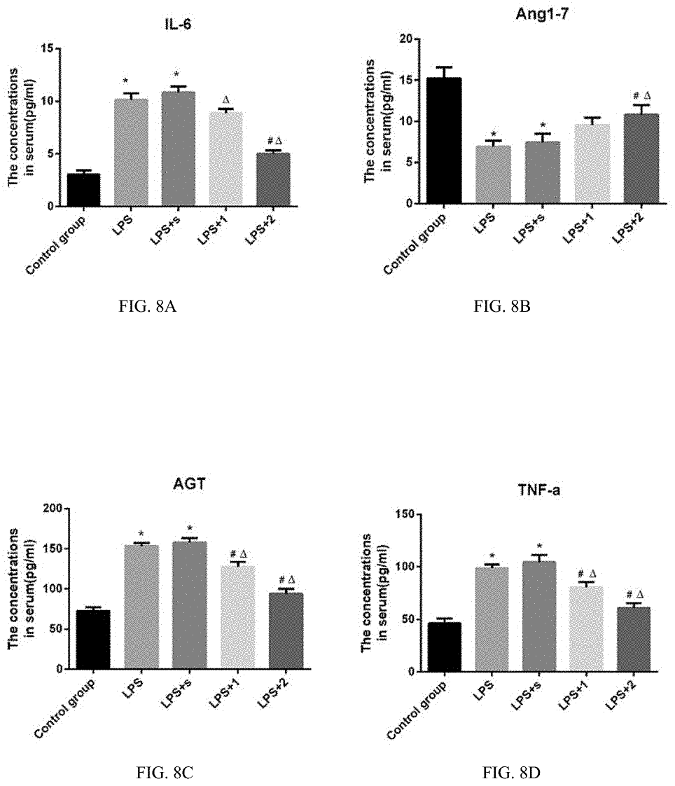

Compared with the Control group, the expression levels of IL-6, TNF-a and AngII in the cell culture medium of LPS and LPS+normal serum group are significantly increased (P<0.05), while the expression level of Ang1-7 is significantly decreased (P<0.05). Compared with the LPS+normal serum group, the expression levels of IL-6, TNF-a and AngII in the Tibetan medicine intervention group 1 and the Tibetan medicine intervention group 2 are significantly decreased (P<0.05), while the expression level of Ang1-7 is significantly increased (P<0.05). The expression levels of IL-6, TNF-a and AngII in the Tibetan medicine intervention group 1 (LPS+1) and the Tibetan medicine intervention group 2 (LPS+2) are significantly lower than those in the LPS alone intervention group (P<0.05), while the expression level of Ang1-7 is significantly increased (P<0.05). The anti-inflammatory effect of the Tibetan medicine intervention group 2 is better than that of the Tibetan medicine intervention group 1. Results are presented in FIGS. 8A-8D.

(2) The Role of Tibetan Medicine in LPS-Induced Acute Lung Injury in Mice

1) Grouping of Experimental Animals

The rats are randomly grouped into four groups: control group, acute lung injury group (LPS), LPS+compound Tibetan medicine extract group (LPS+1), and LPS+compound Tibetan medicine extract group (LPS+2). The treatment conditions of each group are as follows:

Control group: equal saline gavage+intraperitoneal saline injection group

Acute lung injury group: equal saline gavage pretreatment for 30 min+intraperitoneal injection of LPS (5 mg/kg)−Intervention group 1: water extracted alcoholic sediment (1 concentration) after gavage pretreatment for 30 min+intraperitoneal injection of LPS (5 mg/kg)

Intervention groups 2: prescription extract (1 concentration) after 30 min pretreatment by gavage+intraperitoneal injection of LPS (5 mg/kg)

2) The Expression Levels of Cytokine IL-6, TNF-a, AGT and Ang1-7 are Detected by ELISA

Compared with the Control group, the expression levels of IL-6, TNF-a and AGT in the lung tissue of LPS-induced acute lung injury group significantly increases (P<0.05), while the expression level of Ang1-7 significantly decreases (P<0.05). Compared to LPS group, the expression levels of IL-6, TNF-a and AGT significantly decreases (P<0.05) in Tibetan medicine intervention group 1 and Tibetan medicine intervention group 2 Intervention Group, while Ang1-7 expression levels significantly increase (P<0.05).

3) HE Staining for Observing the Morphological Changes of Lung Tissue

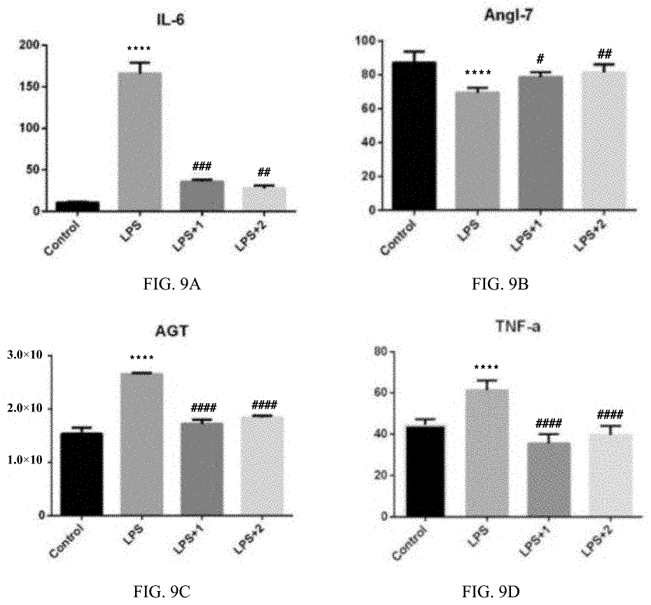

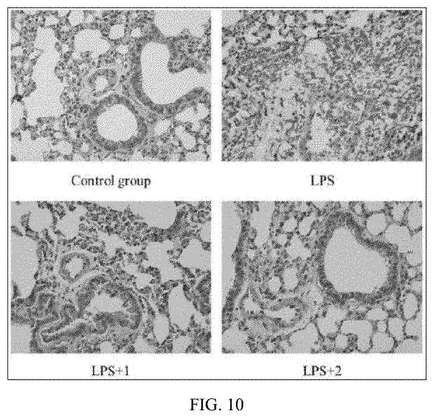

The results of HE staining show that the alveolar tissue in the Control group is structurally intact, and no obvious inflammatory cells are seen in the pulmonary interstitium. In the LPS group, the alveolar structure is damaged significantly, and a large number of inflammatory cell infiltration and severe congestion are observed in the pulmonary interstitium. In the LPS+1 group (Tibetan medicine intervention group 1) and LPS+2 group (Tibetan medicine intervention group 2), the infiltration of inflammatory cells in pulmonary interstitium is significantly reduced, and no significant destruction and congestion in alveolar septum are observed (the results are shown in FIGS. 9A-9D and 10).

To sum up, the interventions of the “Tibetan four-ingredients” extract group all have a protective effect on LPS-induced lung injury, and Tibetan Medicine (prescription extraction) group 2 is better than Tibetan Medicine (prescription extract) group 1 (water extraction and alcohol precipitate). As can be seen, the present application makes a significant omission of medicinal materials and is also able to obtain superior anti-inflammatory as well as lung damage repair effects, which are more beneficial to patients.

What is claimed is:

1. A lung-treating mixture, consisting essentially of extracts of a raw material consisting of Rhododendron anthopogonoides, Gentiana flower, Berberis bark and Herba Corydalis.

2. The lung-treating mixture according to claim 1, wherein the Rhododendron anthopogonoides, the Gentiana flower, the Berberis bark and the Herba Corydalis of the raw material are in parts by weight as that: 100-600 parts of Rhododendron anthopogonoidesoides, 60-120 parts of Gentiana flower, 60-120 parts of Herba Corydalis, and 100-550 parts of Berberis bark.

3. The lung-treating mixture according to claim 1, wherein a medicinal part of the Rhododendron anthopogonoidesoides is flowers and/or leaves, which are flowers and/or leaves of Rhododendron cephalanthum Franch. of Ericaceae family;
wherein a medicinal part of Gentiana flower is flowers, which are flowers of Gentiana szechenyii Kantiz. of Gentianaceae family;
wherein a medicinal part of Herba Corydalis is whole plant, which is whole plant of Corydalis mucronifera Maxim. of Papaveraceae family; and
wherein a medicinal part of Berberis bark is bast, which is inner bast of stem or root of Berberis dasystachya Maxim. of Berberidaceae family.

4. The lung-treating mixture according to claim 1, wherein the extracts comprise volatile oil of the Rhododendron anthopogonoidesoides and water extracts of the raw material.

5. A use of the lung-treating mixture according to claim 1 in preparing a medicament for treating a respiratory disease.

6. The use according to claim 5, wherein the respiratory disease is at least one of a chronic obstructive pulmonary disease (COPD) and acute lung injury.

7. The use according to claim 5, wherein the extracts of the lung-treating mixture are applied in preparing a pharmaceutical preparation for nebulised administration via respiratory system.

8. A medicament for treating a respiratory disease, wherein a pharmaceutically effective amount of the lung-treating mixture according to claim 1 is used as an active ingredient of the medicament.

9. The medicament for treating a respiratory disease according to claim 8, wherein the medicament is in a form of aerosol for nebulised administration through respiratory system; and the medicament comprises the extracts of the lung-treating mixture as the active ingredient, and a nebulizing adjuvant.

10. The medicament for treating a respiratory disease according to claim 9, wherein the medicament is an aerosol for treating at least one of COPD and acute lung injury.
